(19) United States
(12) Patent Application Publication
(10) Pub. No.: US 2018/0366220 A1
(43) Pub. Date: Dec. 20, 2018

(54) Systems and Methods for Pre-Stationing and Regulating Access to Emergency Medical Supplies

(71) Applicant: General Emergency Medical Systems (GEMS), Boston, MA (US)

(72) Inventors: Vincent T. Liao, Cambridge, MA (US); Jeffrey I. Lipton, Medford, MA (US)

(21) Appl. No.: 15/963,556

(22) Filed: Apr. 26, 2018

Related U.S. Application Data
(60) Provisional application No. 62/490,946, filed on Apr. 27, 2017.

(51) Int. Cl. G16H 40/20; G07C 9/00; A61F 17/00; A61M 5/00

(52) U.S. Cl. CPC G16H 40/20; A61M 2205/16; G07C 9/00912; A61F 17/00; A61M 5/002; G07C 2009/00642; G07C 2009/0065; G07C 2209/04; A61M 2205/50; A61M 2205/6036; A61M 2205/3368; A61M 2205/3553; A61M 2205/36; A61M 2205/3606; A61M 2205/14; A61M 2205/6054; G07C 9/00103

(57) ABSTRACT

Systems and methods for pre-stationing and regulating access to emergency medical supplies, by a bystander. The system includes a supply box having medical supplies, a processor, a network interface in communication with the processor, and a keycode receiving device for receiving a keycode associated with the supply box, as well as control software in communication with the network interface of the supply box and at least one database for storing the keycode associated with the supply box. The method includes steps for ultimately enabling the bystander to access the medical supplies for use in response to the emergency.

### RELATED APPLICATION

The present application claims priority to and the benefit of U.S. Provisional Application No. 62/490,946, filed Apr. 27, 2017, which is incorporated herein by reference in its entirety.

### FIELD

The present disclosure generally relates to systems and methods for accessing and providing emergency medical supplies.

### BACKGROUND

Often, in an emergency situation, a bystander, victim, or other participant, hereinafter “bystander”, notices the emergency situation, contacts an emergency dispatcher and then receives instructions regarding appropriate action until emergency services such as, for example, fire, police, emergency medical technicians (EMTs), or paramedics arrive. The emergency dispatcher is an authorized person who dispatches emergency medical services (EMS) and is trained to collect information from and direct the bystander with respect to appropriate action during an emergency. Dispatchers are typically 911 dispatch personnel but may be part of a corporate or private emergency response system. The dispatcher may receive communications via telephone, text message, or other electronic communication methods. Typically, the dispatcher can receive phone calls and voice communication from the bystander. Each dispatcher has a zone of control. Within that zone they are responsible for fielding any emergency situation that occurs.

The scope of possible action taken by the bystander in emergency situations is limited by 1) the bystander's previous training and 2) the bystander's access to medical supplies. Dispatchers are often able to provide commands to the bystander in the event that the bystander has insufficient knowledge for executing appropriate actions during the emergency situation. However, absent access to medical supplies, the bystander is still substantially limited with respect to the scope and quality of aid rendered.

### SUMMARY

The present disclosure provides systems and methods for pre-stationing and regulating access to emergency medical supplies, by a bystander. The system includes a supply box having a processor, a network interface in communication with the processor, a temperature monitoring system, and a keycode receiving device for receiving a keycode associated with the supply box; a first server hosting dispatch control software in communication with the network interface of the supply box; a second server hosting restocking control software in communication with the network interface of the supply box; and at least one database for storing the keycode associated with the supply box. The method for regulating access to emergency medical supplies by a bystander, comprising the steps of maintaining a supply box containing the medical supplies; contacting an emergency dispatcher in the event of an emergency; identifying the emergency where the medical supplies are needed; locating the bystander; determining an estimated speed and time of EMS services, and the type of emergency; comparing the estimated time for the EMS response to a travel time from the bystander to the supply box; directing the bystander to the location of the supply box if the travel time therebetween is less than the estimated EMS response time; and providing the authorized use keycode to the bystander who may then access the medical supplies therein for use in response to the emergency.

### BRIEF DESCRIPTION OF THE DRAWINGS

The presently disclosed embodiments will be further explained with reference to the attached drawings, wherein like structures are referred to by like numerals throughout the several views. The drawings shown are not necessarily to scale, with emphasis instead generally being placed upon illustrating the principles of the presently disclosed embodiments.

FIG. 1A is a perspective view of an embodiment of a medical supply box according to the present disclosure, including the components and contents thereof;

FIG. 1B is a rear view of the medical supply box shown in FIG. 1A;

FIG. 1C is a top plan view of the medical supply box shown in FIG. 1A, with the cover removed for illustrative purposes;

FIG. 1D is a perspective view of the medical supply box shown in FIG. 1C;

FIG. 1E is a sectional view of the medical supply box shown in FIG. 1D, taken along the lines E-E;

FIG. 1F is a perspective view of the medical supply box shown in FIG. 1C, with the front plate removed for illustrative purposes;

FIG. 2 provides a flow chart of a dispatch method of the present disclosure; and

FIG. 3 provides a flow chart of a restocking method of the present disclosure.

While the above-identified drawings set forth presently disclosed embodiments, other embodiments are also contemplated, as noted in the discussion. This disclosure presents illustrative embodiments by way of representation and not limitation. Numerous other modifications and embodiments can be devised by those skilled in the art which fall within the scope and spirit of the principles of the presently disclosed embodiments.

### DETAILED DESCRIPTION

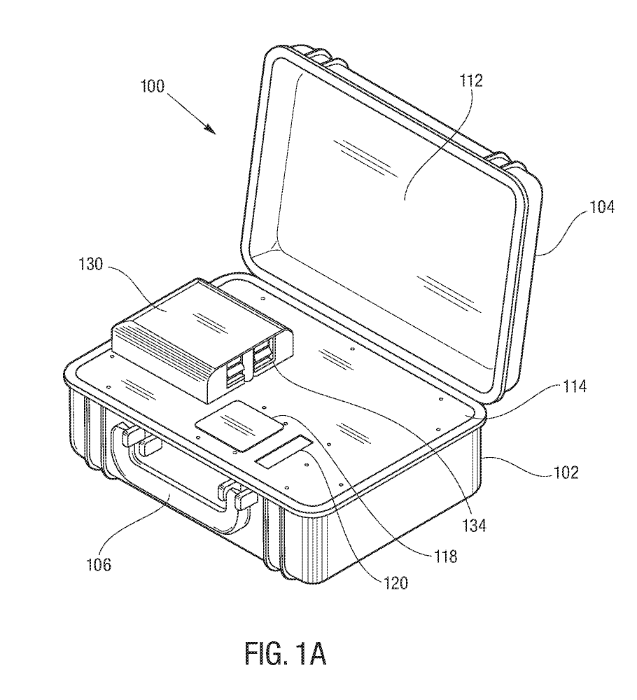

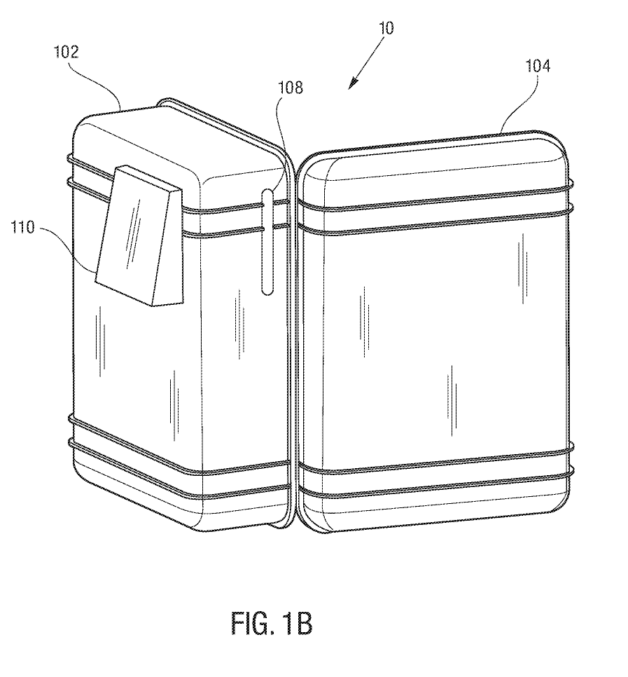

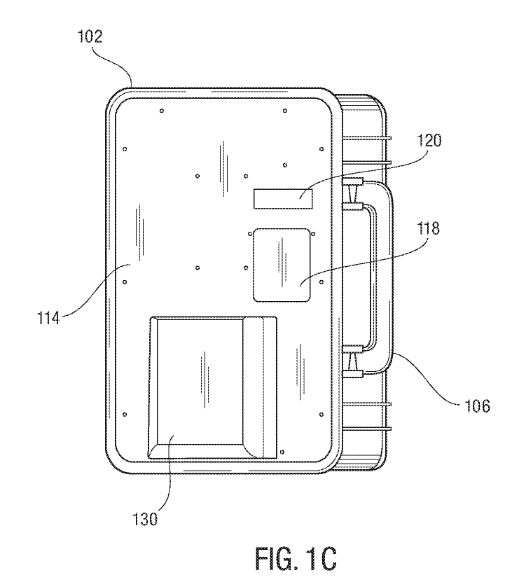

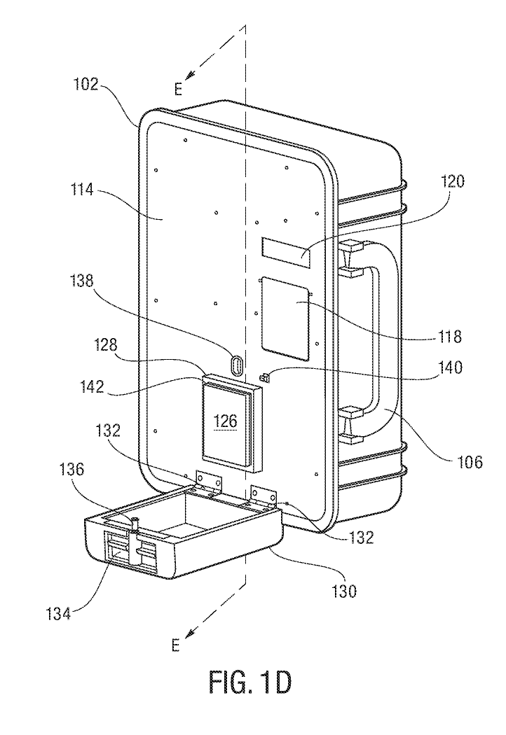

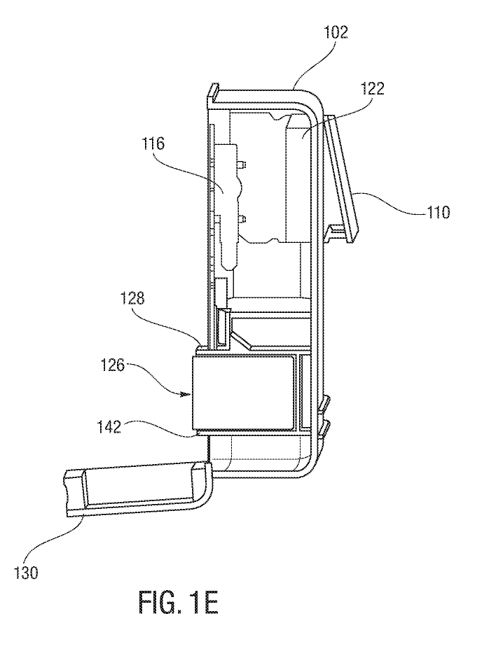

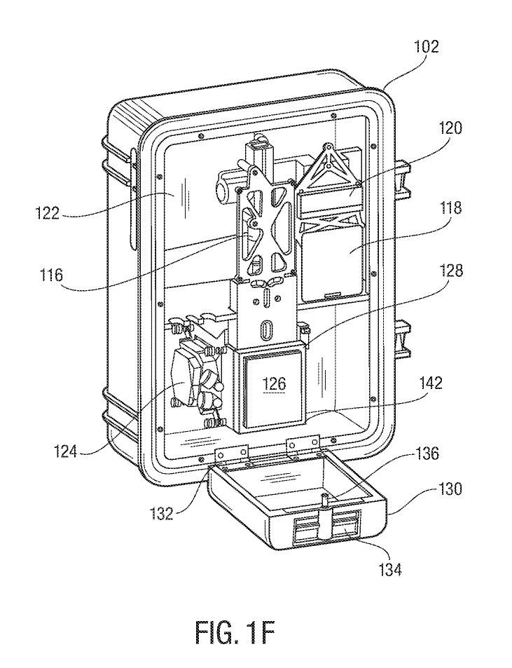

The systems and methods described herein permit the dispatcher to selectively provide the bystander with access to medical supplies, thereby expanding the scope of action taken by the bystander between contacting the emergency dispatcher and the arrival of emergency services. In particular, described herein are methods and systems for pre-stationing and regulating access to emergency medical supplies. The system can include one or more supply boxes (see FIGS. 1A-1F, further discussed below), dispatch control software, restocking control software, and at least one database. The system, in accordance with various embodiments, allows a dispatcher to receive communications from the bystander (or victim or other participant in an emergency situation, hereinafter “bystander”), and provide the bystander access to one of the one or more supply boxes for obtaining emergency medical supplies contained within the supply box.

FIGS. 1A-1F illustrate a supply box 100 having a body, or container, 102 and a detachable cover 104 therefor. The exterior of the container 102 includes a handle 106, an air intake 108 and a vent 110, which are further discussed below. The cover 104 includes a display area 112 on its inner surface, and may be used to present instructions, advertisements and/or other graphical information. A front plate 114 is securely positioned within the container 102 to maintain the various components contained therein, as further discussed below.

The supply box 100 can include a connection to the Internet, an electronic lock 116, and a device for receiving a keycode. The keycode receiving device can, as a non-limiting example, be a keypad 118 with an associated digital display (e.g., LCD) 120. Alternatively, the keycode receiving device can be a touch screen, a RFID reader, a NFC reader, a wireless communication mechanism, an audio receiver, any other digital medium, or combinations thereof. The keycode receiving device allows the possessor of the keycode to unlock the box 100. In some embodiments, two types of keycodes can be used: 1) an authorized use keycode and 2) servicing keycodes. Authorized use keycodes can be provided to bystanders for accessing the contents of the box. Servicing keycodes can permit a person (e.g., box service personnel) to access the box for changing the contents of the box 100.

In some embodiments, the box 100 can include a temperature monitoring system. In some embodiments, the box can include a heating system, a cooling system, or combinations thereof. By use of the temperature monitoring, heating, and cooling systems the box can preserve and protect the contents of the box from exposure to weather, environmental hazards or contaminants, excessive heat, or excessive cold. The air intake 108 and a vent 110 are components of at least some of these systems, along with a ventilation and heat exchange 122 and liquid cooler (e.g., a Peltier cooler or thermoelectric heat pump) 124.

Within the container 102 of the box 100, medical supplies 126 (e.g., in a pouch or other secure, sterile container) are contained within a frame 128 and covered and protected by a lid 130 that is attached to the front plate 114 by hinges 132. Non-limiting examples of medical supplies 126 include bandages, medications (e.g., an epinephrine autoinjector (sold under the trademark EPIPEN) and Naloxone (sold under the trademark NARCAN)), and/or durable equipment such as a scissors or tourniquet. The lid 130 includes a latch 134 with a locking latch pin 136. A latch hole 138 formed in the front plate 114 is dimensioned to securely and releasably engage the locking latch pin 136 of the lid 130 to secure the medical supplies 126 thereunder and within the frame 128. A contact sensor 140 may also be housed in the front plate 114, to detect whether the lid 130 is opened and the medical supplies 126 are exposed. The frame 128 may be made of a durable metal material, and include a metal container 142 therein for housing the medical supplies 126.

The box 100 can also include a processor such as a microcontroller or microprocessor and/or a transceiver for providing electronic communication with dispatch control software, the dispatcher, the restocking control software, and/or the databases. In some embodiments the box 100 can further include a memory for storing the date and set conditions for the contents (e.g., emergency medical supplies) of the box. In some embodiments, two or more of boxes 100 can be connected together to share communications and energy resources. In some embodiments, one or more of the boxes, one or more of the databases, one or more servers hosting the dispatch control software, one or more servers hosting the restocking control software, and/or combinations thereof can be in communication via a public mobile network and/or via the Internet. In some embodiments, one or more of the boxes, one or more of the databases, one or more servers hosting the dispatch control software, one or more servers hosting the restocking control software, and/or combinations thereof can be networked together to form a local network and/or a secured private network.

Electrical energy can be supplied to each box 100 using, for example, power from a power grid, solar power generated by solar panels mounted to or integral with each box. The system can further include one or more primary batteries for providing power to the box 100. Furthermore, the processor, in some embodiments, can include a separate backup battery for providing communications and temperature monitoring in the event the primary power supply is disrupted. In some embodiments, the heating and cooling systems can also include a backup battery for providing temperature control in the event the primary power is disrupted. Such redundant power sources enable the system to operate for extended periods of time when there is no energy input into the device (e.g., due to a grid power outage).

The box 100 can, via the temperature monitoring system, periodically or continuously monitor the temperature conditions of its contents in order to prevent damage to the contents. In accordance with various embodiments, the box 100 is equipped with a temperature control system, including heating and cooling elements (i.e., the air intake 108, vent 110, heat exchange 122 and liquid cooler 124 identified above) whereby the box can use a controller to regulate the internal temperature of the box and, therefore, the contents of the box. In the event that the contents of the box 100 fall outside of a specified temperature range, the contents can be identified as inactive, and the box can enter an inactive state. In the inactive state the box 100 will not accept any authorized use keycodes and may deactivate the temperature control system. In the inactive state, only servicing keycodes will be accepted for unlocking the box 100 when the box is inactive. In some embodiments, the inactive box 100 can transmit its inactive status to an interested party so as to notify the interested party of the inactive status and accompanying need for service and restocking.

For servicing the box 100, a multiple keycode system can be used. The keycodes can be presented to the processor via a network connection, the keycode access system, or a serial port. In some embodiments, multiple types of service keycodes can be used. For example, some keycodes can strictly permit the user to unlock and/or re-lock the box for replacement, removal, or addition of contents to the box while other keycodes can, for example, permit the user to change the conditions of acceptability for the contents (e.g., temperature, expiration date), access the temperature control systems for repair or replacement, and/or reprogram the processor, the temperature monitoring system, or other elements of the box. In some embodiments, each service task may have a unique keycode for entering a different servicing mode. In each servicing mode, a standard set of keycodes can be accepted for performing specific actions. This family of keycodes can be referred to as the service keycodes. When being serviced, the box 100 will typically be opened (i.e., the cover 104 and lid 130 in their open positions) and the contents (i.e., medical supplies 126) will typically be replaced. In general, upon completion of the service (e.g., once the new contents (i.e., the medical supplies 126) are inserted and the lid 130 and cover 104 are reclosed and locked) the temperature control systems can be reactivated.

Once the box 100 again achieves a condition of acceptable operating conditions (e.g., temperature), it can return its state to active, and will again accept authorized use keycodes. The box then automatically (or the service personnel manually) can transmit an identification of the new contents of the box to the control software and databases. The contents can, in some embodiments, include an RFID chip for identification thereof. The RFID chip, when interrogated by an RFID reader, can provide identifying information regarding the contents as well as, in some embodiments, additional information such as expiration date, manufacturing date, manufacturing location, etc. In some embodiments, the RFID reader can be included in the box. In some embodiments, the RFID reader can be a separate unit (e.g., a handheld RFID reader) for use by the service personnel. In some embodiments, the box can transmit the identifying information acquired by the RFID reader to the control software and/or the databases on behalf of the servicing agent.

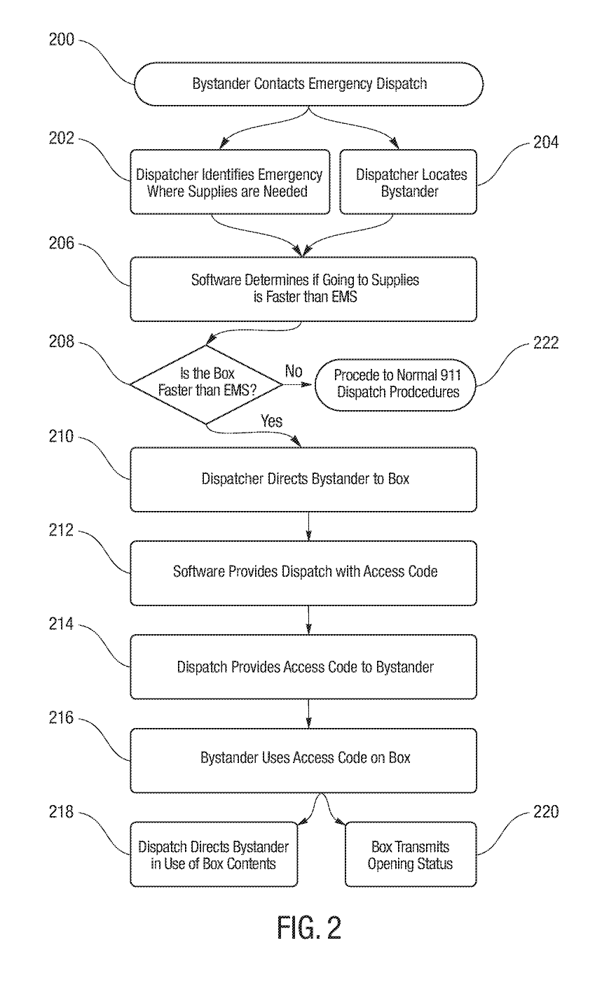

The system operates by pre-stationing emergency medical supplies 126 in boxes 100 throughout a zone of control for an emergency dispatcher, before an emergency occurs. In the event of an emergency, the procedure in the flowchart of FIG. 2 is followed to authorize the bystander (or other participant) to access the emergency medical supplies 126 inside of the box 100, to ultimately deploy them in connection with the emergency. First, a bystander contacts the emergency dispatcher to report the emergency 200. The dispatcher identifies the emergency where the medical supplies are needed 202, and locates the bystander 204. The control software determines the estimated speed and time of EMS services and location of the bystander, and the type of emergency 206. First an estimated time for the EMS response is determined based on distance to nearest available ambulance or database of previous response times and estimates of response times. The nearest active box with supplies is then identified and loaded based on distance. An estimate of the speed and travel time of a bystander from that location is used to determine the time to the box. If no estimate is available then a default speed which is preset is used. If the time to the box is less than that of an EMS response 208 (i.e., for example, less than half the time of the EMS response), then the dispatcher then directs the bystander to the location of the box 210, and the software provides the authorized use keycode to the dispatcher 212. The dispatcher then provides the use keycode to the bystander 214 with instructions on the use of the keycode, who uses the keycode on the box 216. The dispatcher then directs the bystander to take the contents of the box to the person in distress, and to use the contents of the box (i.e., the medical supplies) 218. The box also transmits that it has been opened (e.g., via the contact sensor 140) 220. If, on the other hand, the time to the box is greater than that of an EMS response, then the dispatcher follows normal 911 emergency procedures 222, e.g., by alerting EMS/ambulance/police/firefighters or other responders.

In the event of a power outage at the dispatch location, a paper default for the box can be pre-printed for that day, The dispatcher, unable to use the software, then uses his or her best judgment regarding whether to send the caller to the box, and crosses the box off of the paper list to prevent others from sending a person to the same location. In the event of a mass scale emergency, a keycode can be sent over the Internet to several boxes in an area to unlock them.

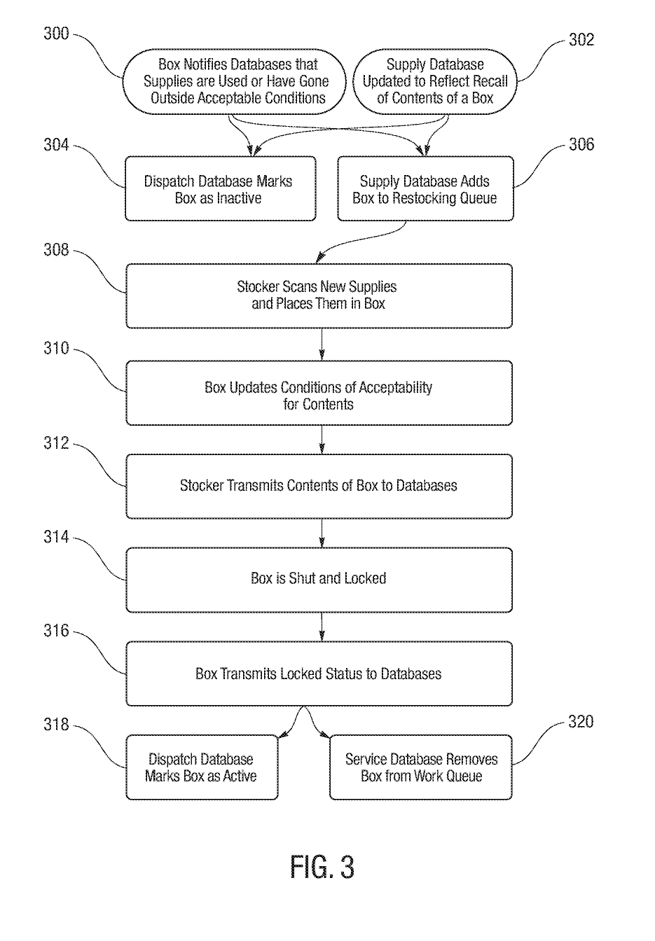

Now referring to the flowchart of FIG. 3, when a box is marked in the inactive state 300, 302, it transmits to two control systems, the dispatch control software and the servicing control software 304. When the dispatch control software receives an inactive state notification from a box or from the service provider, that box is removed from the calculation of the nearest possible box for an emergency. When the dispatch control software receives notification that a box's contents are used or out of acceptable conditions, the box is added to a work queue 306. A serving agent then receives the queue and proceeds to scan the new medical supplies and restock the box with them 308, whereupon and the box updates the contents 310. The agent then transmits same to the databases 312, and closes and locks the box 314. The box then transmits its locked status to the databases 316, resulting in the box being marked active 318 and removed from the work queue 320.

The acceptability conditions for the contents of a box include but are not limited to a maximum and minimum temperature, and a time of expiration. Should the contents of the box fall outside of those ranges, than the contents are deemed inactive and cannot be accessed using an authorized use keycode.

Authorized use keycodes can be provided from a prepared list or can be provided from a calculation process. For example, each box can be assigned a unique identifier. That identifier, along with other information such as, for example, the date and local temperature can be used to generate a keycode both on the box and in the dispatch control software. The keycode can also be generated, for example, in the servicing control software. It will be apparent in view of this disclosure that any other suitable method (e.g., other cryptographic methods) may be employed for generating and accepting keycodes.

As will be understood by those familiar with the art, the invention may be embodied in other specific forms without departing from the spirit or essential characteristics thereof. Likewise, the particular naming and division of the modules, features, attributes, methodologies, managers and other aspects are not mandatory or significant, and the mechanisms that implement the invention or its features may have different names, divisions and/or formats. Furthermore, as will be apparent to one of ordinary skill in the relevant art, the modules, features, attributes, methodologies, managers and other aspects of the invention can be implemented as software, hardware, firmware or any combination of the three. Of course, wherever a component of the present invention is implemented as software, the component can be implemented as a standalone program, as part of a larger program, as a plurality of separate programs, as a statically or dynamically linked library, as a kernel loadable module, as a device driver, and/or in every and any other way known now or in the future to those of skill in the art of computer programming. Additionally, the present invention is in no way limited to implementation in any specific programming language, or for any specific operating system or environment.

All patents, patent applications, and published references cited herein are hereby incorporated by reference in their entirety. It should be emphasized that the above-described embodiments of the present disclosure are merely possible examples of implementations, merely set forth for a clear understanding of the principles of the disclosure. Many variations and modifications may be made to the above-described embodiment(s) without departing substantially from the spirit and principles of the disclosure. It can be appreciated that several of the above-disclosed and other features and functions, or alternatives thereof, may be desirably combined into many other different systems or applications. All such modifications and variations are intended to be included herein within the scope of this disclosure, as fall within the scope of the appended claims.

What is claimed is:

1. A system for pre-stationing and regulating access to emergency medical supplies comprising:
a supply box including:
a processor,
a network interface in communication with the processor,
a temperature monitoring system, and
a keycode receiving device for receiving a keycode associated with the supply box;
a first server hosting dispatch control software in communication with the network interface of the supply box;
a second server hosting restocking control software in communication with the network interface of the supply box; and
at least one database for storing the keycode associated with the supply box.

2. The system of claim 1, wherein the supply box further includes a heating system, a cooling system, or a combination thereof.

3. The system of claim 1, wherein the supply box further includes medical supplies, and a frame and lid for containing the medical supplies.

4. The system of claim 3, wherein the supply box further includes a contact sensor proximate the medical supplies and lid, so as to detect whether the lid is opened and the medical supplies are exposed.

5. The system of claim 1, wherein the keycode receiving device is selected from a group consisting of a keypad, a touch screen, a RFID reader, a NFC reader, a wireless communication mechanism, an audio receiver, and combinations thereof.

6. The system of claim 1, further comprising at least one power supply.

7. The system of claim 6, wherein the at least one power supply includes a backup power supply.

8. A system for pre-stationing and regulating access to emergency medical supplies comprising:
a supply box including:
a processor,
a network interface in communication with the processor,
a keycode receiving device for receiving a keycode associated with the supply box;
a first server hosting dispatch control software in communication with the network interface of the supply box; and
at least one database for storing the keycode associated with the supply box.

9. The system of claim 8, wherein the supply box further includes a temperature monitoring system.

10. The system of claim 8, wherein the system further comprises a second server hosting restocking control software in communication with the network interface of the supply box.

11. The system of claim 8, wherein the supply box further includes a heating system, a cooling system, or a combination thereof.

12. The system of claim 8, wherein the supply box further includes medical supplies, and a frame and lid for containing the medical supplies.

13. The system of claim 8, wherein the keycode receiving device is selected from a group consisting of a keypad, a touch screen, a RFID reader, a NFC reader, a wireless communication mechanism, an audio receiver, and combinations thereof.

14. The system of claim 8, further comprising at least one power supply.

15. A method for regulating access to emergency medical supplies by a bystander, comprising the steps of:
maintaining a supply box containing the medical supplies;
contacting an emergency dispatcher in the event of an emergency;
identifying the emergency where the medical supplies are needed;
locating the bystander;
determining an estimated speed and time of EMS services, and the type of emergency;
comparing the estimated time for the EMS response to a travel time from the bystander to the supply box;
directing the bystander to the location of the supply box if the travel time therebetween is less than the estimated EMS response time; and
providing the authorized use keycode to the bystander, whereby the bystander may access the medical supplies therein for use in response to the emergency.

16. The method of claim 15, wherein the estimated EMS response time is determined based on distance to a nearest available ambulance.

17. The method of claim 15, wherein the estimated EMS response time is determined based on previous response times and estimates of response times.

18. The method of claim 15, wherein the travel time from the bystander to the supply box is a preset default speed.

19. The method of claim 15, wherein the providing step includes providing the authorized use keycode to the dispatcher, and providing the use keycode from the dispatcher to the bystander.

20. The method of claim 15, wherein the maintaining step includes
monitoring the status of the supply box and the medical supplies therein;
identifying the supply box as inactive if the medical supplies are used or out of acceptable conditions;
adding the supply box to a work queue;
restocking the supply box with new medical supplies; and
identifying the supply box as active.
